(19) United States
(12) Patent Application Publication
(10) Pub. No.: US 2001/0017134 A1
(43) Pub. Date: Aug. 30, 2001

(54) Conduit for connecting a fluid transfer device to a patient

(75) Inventor: Pontus Von Bahr, Enskede (SE)

Correspondence Address: SCHIFF HARDIN & WAITE, 6600 SEARS TOWER, 233 S WACKER DR, CHICAGO, IL, 60606-6473, US

(73) Assignee: Siemens Elema AB

(21) Appl. No.: 09/778,508

(22) Filed: Feb. 7, 2001

(30) Foreign Application Priority Data
Feb. 24, 2000 (SE) 0000605-6

(51) Int. Cl. A62B 7/00; A61M 16/00

(52) U.S. Cl. 128/204.18

(57) ABSTRACT

A conduit connectible in a fluid transfer system to provide for the through flow of a fluid moving between a transfer device and internal patient includes a transmission line incorporated therewith having a distal end for signal communication with a signal generator and a proximal end connected to a terminal on a mating surface of a female connector and arranged thereon to establish a signal transmission path with a terminal on a mating surface of a second conduit, such as a port of the fluid transfer device, as the mating surfaces engage to establish fluid communication between the conduit and the port.

### BACKGROUND OF THE INVENTION

1. Field of the Invention

The present invention relates to a conduit used in a fluid flow path connecting a fluid transfer device to a patient and to a fluid transfer system incorporating the same.

2. Description of the Prior Art

Very often a fluid transfer device which is used in patient care, such as a liquid ventilator which transfers a breathing liquid to and from a patient, a gas ventilator or respirator which transfers gas to and from a patient, or a dialysis machine which transfers blood to and from a patient, needs to communicate with external elements such as sensors or valves connected to a fluid flow path linking the device to the interior of a patient.

Such communication may be necessary to enable the device to control an external valve in order to regulate the fluid flow within the flow path. Alternatively or additionally, such communication may be necessary to provide the device with information from sensors in communication with the fluid which is useable within the device for control purposes or which is useable in a display system to provide a perceptible indication of the efficacy of the care being provided.

This requires that, in addition to the fluid conduits which form the fluid flow path, one or more transmission lines, such as electrical wires or optical cables, be provided along which signals may be transmitted. These lines often produce additional installation work for the user, more difficult transportation of the patient, and an increased risk of accidental user or patient interaction with the lines which might cause involuntary disconnection of the external elements or a potential heath hazard.

It is known to provide a fluid conduit having incorporated therein an optical fiber transmission line having a proximal end for signal communication with a TV camera and a distal end for receiving light reflected from a region to be imaged using the camera.

### SUMMARY OF THE INVENTION

It is an object of the present invention to alleviate at least one of the problems associated with the presence of such transmission lines.

This object is achieved in accordance with the principles of the present invention in a conduit having a hollow, flexible tubular body with a first end and a second end, an electrical transmission line disposed in the body proceeding between the first and second ends and having a first set of terminals disposed at the first end and a second set of terminals disposed at the second end, and an interface permanently attached at the first end having a surface at which the first terminals are disposed, the interface being adapted for mating with a complementary interface to simultaneously provide an electrical connection to the first terminals as well as a fluid coupling to the hollow body.

By providing a transmission line, for example an optical or an electrical transmission line, which is incorporated with the fluid conduit and which is terminated at one or both of the proximal and the distal ends of the conduit by an interface adapted to establish a concomitant signal and fluid coupling of the conduit with another conduit within a fluid transfer system then the potential for accidental user or patient interaction with the line is reduced, as is the installation work for a user and the possibility of failing to establish a signal connection.

The line is incorporated so as to substantially maintain the flexibility of the conduit, such as for example by being helically wound to follow the contours of a “concertina” hose that is often used as a flexible conduit within a gas flow path of a ventilator system.

The above object is also achieved in accordance with the invention in a fluid transfer device employing a conduit as described above, connected to a fluid transfer device.

The transfer device includes a port through which fluid may be transferred between the interior and the exterior of the device, having, at an externally accessible surface, a terminal connected to the processing unit, and an interface is provided for establishing both a coupling of the transmission line with the terminal of the port and a fluid coupling of the proximal end of the conduit with the port. In this manner both signal and fluid communication are established between the conduit and the device in a single operation.

A detector may be connected to the input terminal of the transfer device and for detecting the connection of the sensor to the transmission line. This allows an automatic confirmation of a correct coupling to be made or an automatic re-configuration of the system to accept signals from a sensor connected using conventional transmission lines.

### DESCRIPTION OF THE DRAWINGS

FIG. 1 is a schematic representation of a conduit according to the present invention.

FIG. 2 is a schematic representation of a further embodiment of a conduit according to the present invention.

FIG. 3 shows elements of a fluid transfer system according to the present invention incorporating the conduits of FIGS. 1 and 2.

### DESCRIPTION OF THE PREFERRED EMBODIMENTS

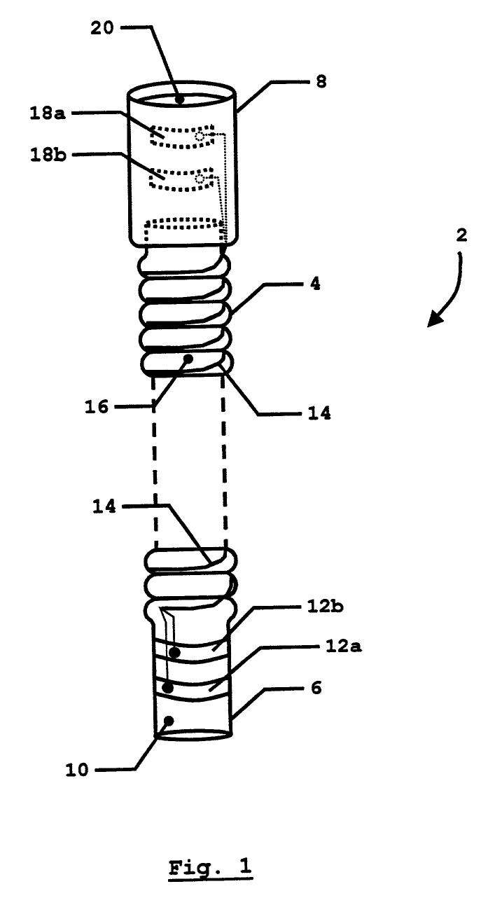

As shown in FIG. 1, a conduit 2 according to the present invention is formed by a length of flexible gas tubing 4 having, at opposite ends, a male interface 6 and a female interface 8 for interconnecting with other gas conduits.

The male interface 6 may, for example, be formed of a rigid plastic material over which a female sealing piece of another conduit can befit, and has two electrical contacts 12a and 12b on an external mating surface 10. These contacts 12a and 12b are connected to respective electrical wires of a two-core cable 14. The cable 14 is helically wound around an external surface 16 of the gas tubing 4 and opposite ends of the wires are connected to respective electrical contacts 18a and 18b which are located on an internal mating surface 20 of the female interface 8. The female interface 8 can be formed of a flexible rubber material to form a gas seal when push-fit with a cooperating male interface of another conduit. In this manner, an electrical signal transmission path is provided along the length of the tubing 4, between corresponding pairs of externally accessible contacts 12a, 18a and 12b, 18b.

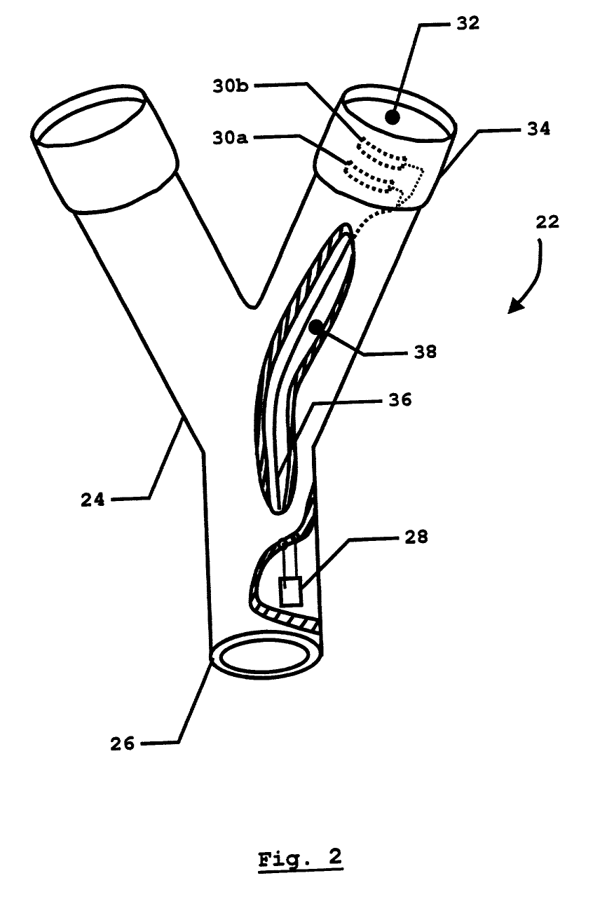

As shown in FIG. 2, a conduit 22 according to the present invention has a rigid Y-piece connector 24. Located within the common stem-section 26 is a sensor 28, such as a solid state pressure transducer or a flow meter. Electrical contacts 30a and 30b are located on an internal mating surface 32 of an interface 34 which is here shown in the form of a female connector adapted to form a gas seal when mated with a cooperating male connector of another conduit. A cable 36 is provided to establish an electrical signal transmission path between the sensor 28 and the contacts 30a and 30b and is incorporated with the conduit 22 within a channel 38 in a side wall of the Y-piece connector 24.

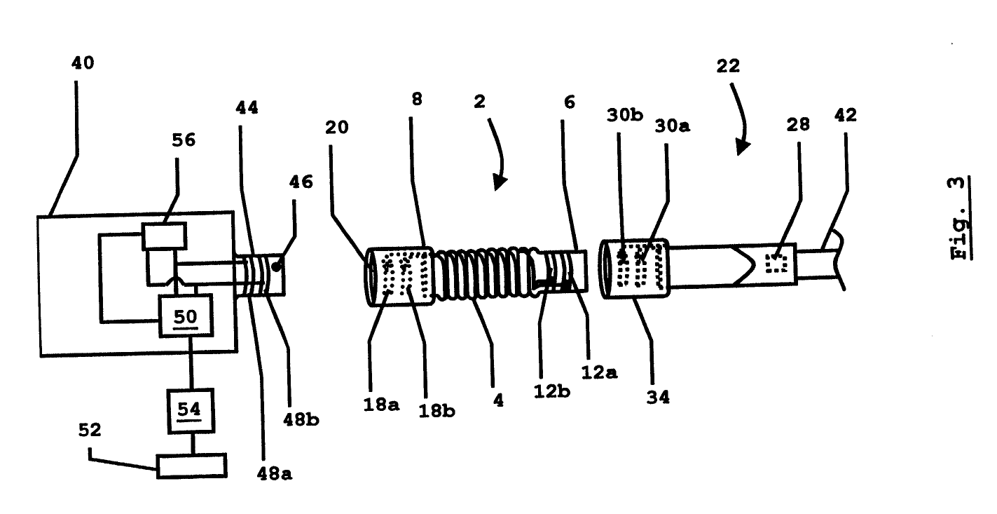

An example of a fluid transfer system according to the present invention for the transfer of gas between the airways of a patient (not shown) and a fluid transfer device (ventilator 40) of the system is shown in FIG. 3. Conduits 2, 22 are interconnectable with an endotracheal tube 42 (shown in part) to form a flow path for gas passing between the ventilator 40 and a patient's airways (not shown) into which the endotracheal tube 42 will be inserted in use.

The ventilator 40 includes a gas port 44 which has, on an external mating surface 46, two electrical contacts 48a, 48b intended for connection with the contacts 18a and 18b provided on the inside of the female interface 8 at a proximal end of the flexible tubing 4. The contacts 48a and 48b, and 18a and 18b, are disposed on their respective surfaces 46, 20 such that when the female interface 8 is push fit over the port 44 to thereby form a gas tight connection, the contacts 48a and 48b and 18a and 18b will establish an electrical connection. Preferably one or both sets of contacts 48a and 48b and 18a and 18b are provided as rings around the periphery of the corresponding mating surface 46. This enables signal communication to be established between the sets of contacts 48a and 48b and 18a and 18b regardless of the rotational orientation of the conduit 2.

Similarly, when the male interface 6 at the distal end of the tubing 4 is interconnected with the female interface 34 to establish a gas tight connection, then an electrical connection is automatically established between the electrical contacts 12a and 12b and 30a and 30b (either or both of which may be formed as ring contacts. Thus, a signal path between a sensor 28, here within the Y-piece conduit 22, and a processing unit 50, here within the ventilator 40 and connected to the contacts 48a and 48b of the port 44, is automatically established as the flow path for connecting the ventilator 40 to a patient's airways is established.

The processing unit 50 functions to control the operation of the ventilator 40 to regulate gas transfer so as to provide a desired breathing pattern, as is known in the art of mechanical ventilation, for example to maintain flow parameters as set by a user via an interface 52. Also as common in the art, the processing unit may process the output from the sensor 28 to provide a visible indication on a display 54 of monitored properties of the gas, which may be used to determine the efficacy of the ventilation therapy being provided by the ventilator 40.

A detector 56 may be connected to the contacts 48a and 48b in order to detect whether a sensor is connected, which may also indicate whether the gas/electrical interconnections between the port 44 and the interface 8 at the proximal end of the tubing 4, and between the interface 6 at the distal end of the tubing 4 and the interface 34 of the Y-piece conduit 22, have been properly established. This can be done by adapting the detector 56 to monitor the resistance of the transmission path essentially between the sensor 28 and the contacts 48a and 48b of the port 44.

The absence of a sensor 28 or the absence of proper interconnections can be reported to the user via the display 54, as an output either directly from the detector 56 or from the processing unit 50 to which the detector 56 may also be connected. The processing unit 50 may then be configured to inhibit the operation of the ventilator 40 or to use conventional sensor inputs in response to the report from the detector 56.

It will be appreciated by those skilled in the art that the embodiments of the present invention provided above may be modified in a number of ways. For example, the signal transmission lines may be used to provide bidirectional communication, or communication with devices other than sensors, such as flow control valves. Additionally, more than one transmission line may be incorporated with the conduit and the one or more transmission lines may be optical fibers or acoustic waveguides.

I claim as my invention:

1. A conduit comprising:
a hollow, flexible tubular body having a first end and a second end;
an electrical transmission line disposed at said body and proceeding between said first end and said second end and having a first set of terminals disposed at said first end and a second set of terminals disposed at said second end; and
an interface permanently attached at said first end having an interior at which said first terminals are disposed, said interface being adapted for mating with a complementary interface to simultaneously provide an electrical connection to said first terminals and a fluid coupling to said hollow body.

2. A conduit as claimed in claim 1 wherein said interface comprises a male connector having a mating surface at which said first set of terminals is disposed.

3. A conduit as claimed in claim 1 wherein said interface comprises a female connector having a mating surface at which said first set of terminals is disposed.

4. A conduit comprising:
a hollow tubular body having a first end and a second end;
electrical conductors disposed at said hollow body and proceeding between said first end and said second end;
an interface disposed at said first end having a first set of terminals connected to said electrical conductor and adapted for mating with a complementary interface for producing an electrical connection to said electrical conductors and a fluid connection to an interior of said hollow body; and
a sensor connected to said electrical conductors and disposed at said second end for sensing a parameter relating to fluid flow in said hollow body.

5. A fluid transfer system comprising:
a fluid transfer device;
a conduit connectible in a flow path between said transfer device and a location adapted to provide fluid flow to a patient;
a sensor for monitoring a parameter of said fluid flow and for generating a signal representing said parameter;
a processing unit supplied with said signal which generates an output signal dependent thereon;
a transmission line incorporated with said conduit having a first end connected to said sensor and a second end for connection to said processing unit;
said fluid transfer device having a port for transferring fluid into and out of said fluid transfer device and having an externally accessible surface;
a terminal at said externally accessible surface connected to said processing unit; and
said conduit having an interface permanently attached thereto for mating with said port for simultaneously electrically connecting said transmission line to said terminal and for producing a fluid coupling of said conduit with said fluid transfer device.

6. A system as claimed in claim 5 wherein said interface is a male connector having a mating surface at which said terminal is disposed, and wherein said port is a female connector having a mating surface complementary to said mating surface of said male connector.

7. A system as claimed in claim 5 wherein said interface is a female connector having a mating surface at which said terminal is disposed, and wherein said port is a male connector having a mating surface complementary to said mating surface of said female connector.

8. A system as claimed in claim 5 wherein said fluid transfer device receives said output signal from said processing unit and regulates transfer of said fluid into and out of said fluid device dependent on said output signal.

9. A system as claimed in claim 5 further comprising a detector electrically connected to said terminal at said port for detecting connection of said sensor to said transmission line.
